(19) United States
(12) Patent Application Publication
(10) Pub. No.: US 2025/0362281 A1
(43) Pub. Date: Nov. 27, 2025

(54) METHOD FOR DETERMINING USABILITY OF CEMENT-BASED COMPOSITES

(71) Applicant: SHANDONG UNIVERSITY, Jinan (CN)

(72) Inventors: Fengling ZHANG, Jinan (CN); Kai PANG, Jinan (CN); Jinghang ZHANG, Jinan (CN); Jinglong LI, Jinan (CN); Chenxu YAN, Jinan (CN); Tongchun SU, Jinan (CN); Xiangyu MENG, Jinan (CN); Yupeng CUI, Jinan (CN); Xiaoyu LI, Jinan (CN); Min TANG, Jinan (CN)

(21) Appl. No.: 19/218,709

(22) Filed: May 27, 2025

(30) Foreign Application Priority Data
May 27, 2024 (CN) 2024106649546
Jun. 7, 2024 (CN) 2024107378908

(51) Int. Cl. G01N 33/00; C04B 14/04; C04B 14/48; G01N 33/38

(52) U.S. Cl. CPC G01N 33/0003; C04B 14/048; C04B 14/48; G01N 33/383

(57) ABSTRACT

The present invention provides a method for determining usability of cement-based composites, comprising: regarding cement-based composites with different known components as multiphase materials, respectively measuring Rockwell hardnesses of each phase; calculating an equivalent length based on volume ratio of each phase; acquiring an effective hardness of the cement-based composite through weighted calculation based on the Rockwell hardness and the equivalent length of each phase; measuring penetration resistance of the cement-based composite with different components, and establishing an association relationship between the effective hardness and the penetration resistance; calculating an effective hardness of a cement-based composite with unknown penetration resistance, calculating an evaluation value of the penetration resistance by using the established association relationship; and applying the cement-based composite to intended use thereof when a judging result shows that an applicable result corresponding to the acquired evaluation value is within a threshold range.

### CROSS-REFERENCE TO RELATED APPLICATIONS

The present invention claims priority to Chinese Patent Application No. 202410664954.6, filed with the China National Intellectual Property Administration on May 27, 2024 and entitled “Rockwell Hardness Measurement Method, Effective Hardness, and Application of Cement-based composites”, and priority to Chinese Patent Application No. 202410737890.8, filed with the China National Intellectual Property Administration on Jun. 7, 2024 and entitled “Method for Evaluating Penetration Resistance of Fiber-reinforced Cement-based composites”, which are incorporated herein by reference in their entireties and constitute a part of the present invention for all purposes.

### TECHNICAL FIELD

The present invention belongs to the technical field of physical performance measurement of a building material, and relates to a method for determining usability of cement-based composites.

### BACKGROUND

Information of the BACKGROUND part is merely disclosed to enhance the understanding on the overall background of the present invention, but is not necessarily regarded as acknowledging or implying, in any form, that the information constitutes the prior art known to a person of ordinary skill in the art.

Cement-based composites are man-made materials with widest use range and greatest use quantity in the world, encompassing cement pastes, cement mortars, concretes, engineered cementitious composites, ultra-high-performance concretes, and the like.

Compressive strength is one of most important index parameters of the cement-based composites such as concrete. There has been a consensus for a long time that a higher compressive strength indicates better penetration resistance of the cement-based composites, which is specifically manifested in that a penetration depth of a projectile in the cement-based composites gradually decreases as the compressive strength increases. Actually, the material composition, the microstructure, and the crack propagation of the cement-based composites affect the penetration resistance of the cement-based composites to different degrees. With the progress of concrete technologies, the above association relationship between the penetration resistance and the compressive strength has not been applicable to advanced cement-based composites such as ultra-high-performance concretes (UHPC) and engineered cementitious composites (ECC). Particularly, after the compressive strength exceeds a particular threshold (120 MPa), continuously improving the compressive strength of the cement-based composites cannot further reduce their penetration depth under the action of the high-speed projectile. The compressive strength loses a clear association relationship with the penetration resistance, and the penetration resistance cannot be evaluated using the value of the compressive strength. Therefore, it urgently needs to provide a method for evaluating the penetration resistance of the cement-based composites capable of comprehensively considering the influence of the material composition.

Rockwell hardness is a common performance index for a concrete material, and a test value of the Rockwell hardness may be easily influenced by various factors such as test force, a shape and a size of an indenter, a surface condition of a specimen, and the like. Therefore, strict test conditions and operating procedures need to be followed during a Rockwell hardness test to ensure accuracy and reliability of a measuring result. However, because the composition of the cement-based composites is complex, at present, there is no simple and highly repeatable method for testing the Rockwell hardness and effective hardness of the cement-based composites.

### SUMMARY

By aiming at the limitations in the prior art, an object of the present invention is to provide a method for determining usability of cement-based composites. In the present method, a relationship between an effective hardness value and penetration resistance is first established based on a Rockwell hardness; then, the penetration resistance of the cement-based composites can be fast evaluated by utilizing a measurement result of the Rockwell hardness; thereafter, the application of the cement-based composites to intended use thereof is determined based on an acquired evaluation result value and is performed. The method for measuring the Rockwell hardness provided by the present invention effectively improves the accuracy of a Rockwell hardness measuring result of the cement-based composites, so that an evaluation result is more accurate.

In order to achieve the above objective, the present invention adopts the following technical solutions:

The method for determining usability of cement-based composites includes:

S1: regarding a cement-based composite with different known components as a multiphase material, and dividing each component of the cement-based composite into multiple constituent phases;

S2: acquiring samples of all constituent phases, and respectively measuring a Rockwell hardness of each phase;

S3: calculating an equivalent length of each phase based on a volume ratio of each phase in the cement-based composite;

S4: acquiring an effective hardness of the cement-based composite through weighted calculation based on the Rockwell hardness of each phase acquired through measurement in S2 and the equivalent length of each phase acquired in S3;

S5: measuring penetration resistance of the cement-based composite with different components, and establishing an association relationship between the effective hardness and the penetration resistance based on the effective hardness corresponding to multiple cement-based composites with different known components acquired in S1 to S4;

S6: regarding a cement-based composite with unknown penetration resistance as a multiphase material, calculating an effective hardness of the cement-based composite with unknown penetration resistance according to S1 to S4, and performing calculation using the association relationship acquired in S5 to acquire an evaluation value of the penetration resistance of the cement-based composite with unknown penetration resistance; and

S7: judging a corresponding threshold range of the acquired evaluation value, and applying the cement-based composite to intended use thereof.

The present invention has the following beneficial effects:

1. The present invention provides the method for determining usability of cement-based composites, by the method, the influence of each constituent phase of the materials may be comprehensively considered, and a new method for expressing the penetration resistance of the cement-based composites with the effective hardness is established based on the Rockwell hardness. The penetration resistance (penetration depth and crater diameter) of the cement-based composite with different material compositions and complex performance may be precisely expressed under the failure of corresponding relationship between a compressive strength and the penetration resistance. The relay of an existing cement-based composite on a conventional penetration experiment for penetration resistance evaluation is avoided. Through the evaluation method provided by the present invention, the penetration resistance of the cement-based composite may be fast evaluated. Additionally, the damage to the sample is small, so the completeness of the sample may be maintained, and the reliability and repeatability of a test may be improved.
2. The present invention deep analyzes a relationship between a microstructure and macro-performance of the cement-based composite, the penetration resistance of the cement-based composite is predicted using an advanced material hardness test technology. The method is not only applicable to studies in a laboratory, but also applicable to on-site fast measurement, and provides convenience for material selection and quality control in engineering application. In addition, by optimizing a test process, the present invention significantly improves the measurement efficiency, and reduces the time and cost required by the test, so that the penetration resistance evaluation of the material is more economic and efficient. The use of the present invention is not limited to the performance evaluation of the cement-based composites, and may also be expanded to the performance evaluation of other advanced engineering materials, the penetration resistance of the materials may be more comprehensively understood and utilized, and powerful technical support is provided for further study and development of the penetration resistance of the materials.

### BRIEF DESCRIPTION OF THE DRAWINGS

The accompanying drawings constituting a part of the present invention are used to provide a further understanding of the present invention. The exemplary examples of the present invention and descriptions thereof are used to explain the present invention, and do not constitute an improper limitation to the present invention.

FIG. 1 is a schematic diagram of a Rockwell hardness measuring process in a specific implementation of the present invention.

FIG. 2 is a schematic diagram of a selection manner of pressure test point regions in a specific implementation of the present invention.

FIG. 3 is a result distribution diagram of a Rockwell hardness measuring method in a specific implementation of the present invention.

FIG. 4 is a schematic diagram of a relationship between normalized penetration depths and compressive strengths of cement-based composites in a specific implementation of the present invention.

FIG. 5 is a schematic diagram of an effective length of a cement-based composite in Example 1 of the present invention.

FIG. 6 is a schematic diagram of an association relationship between normalized penetration depths and effective hardnesses of cement-based composites in Example 3 of the present invention.

FIG. 7 is a schematic diagram of an association relationship between crater diameters and indentation depths of the cement-based composites in Example 3 of the present invention.

FIG. 8 is a schematic diagram of an effective length of a cement-based composite in a Comparative example of the present invention.

In the figures: 1, projectile trajectory; 2, projectile; 3, mortar matrix phase; 4, coarse aggregate phase; and, 5, denotes a fiber phase.

### DETAILED DESCRIPTION

It should be noted that, the following detailed descriptions are all exemplary, and are intended to provide further descriptions of the present invention. Unless otherwise specified, all technical and scientific terms used herein have the same meanings as those usually understood by a person of ordinary skill in the art to which the present invention belongs.

It should be noted that the terms used herein are merely used for describing specific implementations, and are not intended to limit exemplary implementations of the present invention. As used herein, the singular form is intended to include the plural form, unless the context clearly indicates otherwise. In addition, it should further be understood that terms “comprise” and/or “include” used in this specification indicate that there are features, steps, operations, devices, components, and/or combinations thereof.

### Example 1

A method for determining usability of cement-based composites is provided, including a Rockwell hardness measuring method.

The method for measuring a Rockwell hardness of cement-based composites includes:

S21: The cement-based composite is cast to acquire three cube sample blocks, each sample block had four side surfaces adjacent to a cast surface, and the sample block is subjected to demoulding, grinding, and cleaning to acquire a sample to be tested in a size of 100×100×100 mm³.

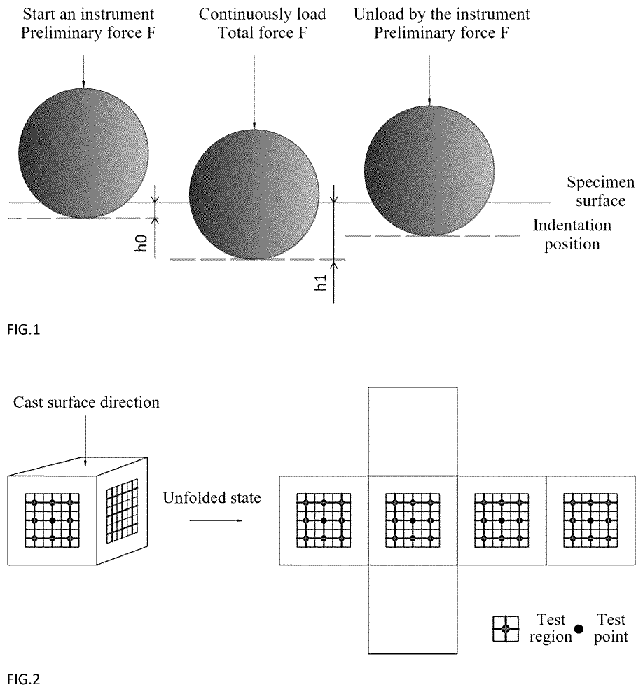

S22: After multiple side surfaces are ground to be flat and smooth, powder remained on the surfaces of the sample is wiped off with alcohol cotton balls to avoid the influence of the powder on a subsequent indentation test. As shown in FIG. 2, multiple lines perpendicular to edges of the side surfaces are drawn on the side surfaces to equally divide each side surface into small square blocks being 20×20 mm, and a total quantity of the small square blocks is 25; and in order to avoid the influence of an edge effect, the middle nine small square block regions are selected to be used as pressure test point regions.

S23: Points are taken from center positions of the small square blocks in the measuring regions to test Rockwell hardnesses, and indentation data of multiple effective points are read.

S24: At least nine effective points are selected from each side surface, so 3×4×9 effective points might be totally acquired from three sample blocks, and an average value of indentation data of at least 100 effective points on three cube sample blocks is taken to be used as the Rockwell hardness of the cement-based composite to solve its standard deviation.

In S23, a method for measuring the Rockwell hardness of a single effective point includes:

The sample is placed onto a platform of a Rockwell hardness test instrument, and is fixed.

A steel ball with a diameter being 1.5875 mm is selected to be used as an indenter, a preliminary force is set, and is preferably 29.42 N, and a total force is set, and is preferably 147.1 N.

The instrument is started, as shown in FIG. 1, the sample is in contact with the indenter until the preliminary force is reached, the preliminary force is maintained for 15 s, and an indentation depth is measured by the instrument and is counted as a base line indentation depth h₀.

The instrument continuously loaded to reach the total force, the total force is maintained for 15 s, and is then unloaded to the preliminary force, the preliminary force is maintained for 15 s, and an indentation depth is measured by the instrument and is counted as a final indentation depth h₁.

The indentation data is a difference value between the final indentation depth and the base line indentation depth of the Rockwell hardness test instrument, the Rockwell hardness H is acquired through calculation, and

$$H = 100 - \frac{{h}_{1} - {h}_{0}}{0.001} .$$

During parameter test in S23, a method for judging the effective points includes: a distance between two indentation centers should be at least 4 times (6.35 mm) of a diameter of a spherical indenter, and should not be smaller than 2 mm; and in addition, a distance between any one indentation center and the edge of the sample should be at least 2.5 times of the diameter of the spherical indenter, and should not be smaller than 1 mm.

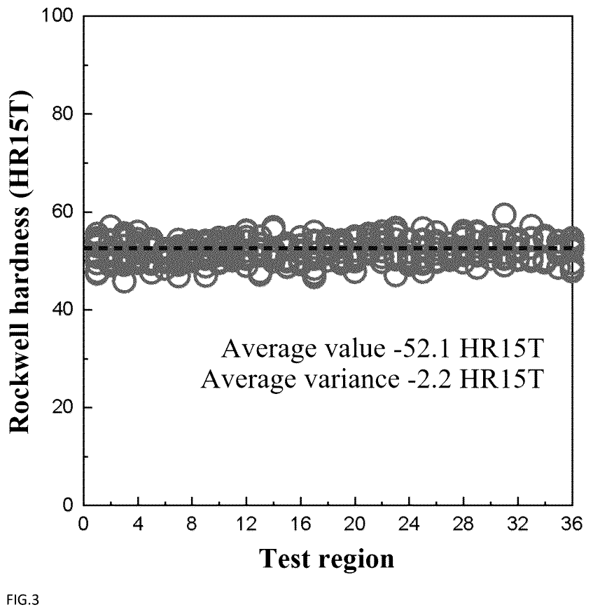

According to the method in the present example, the Rockwell hardness of the cement-based composite with set components is measured, in addition, the Rockwell hardness measurement is performed on the same material according to a random point selecting method, and measuring results of the method of the present example is counted, as shown in FIG. 3. Its variance is solved: a variance value of the method of the present example is 2.2, and a variance value of a random point selecting method is 4.5. It could be seen that the data of the method of the present example is more concentrated, the variance is lower, and the more excellent accuracy and reliability are achieved.

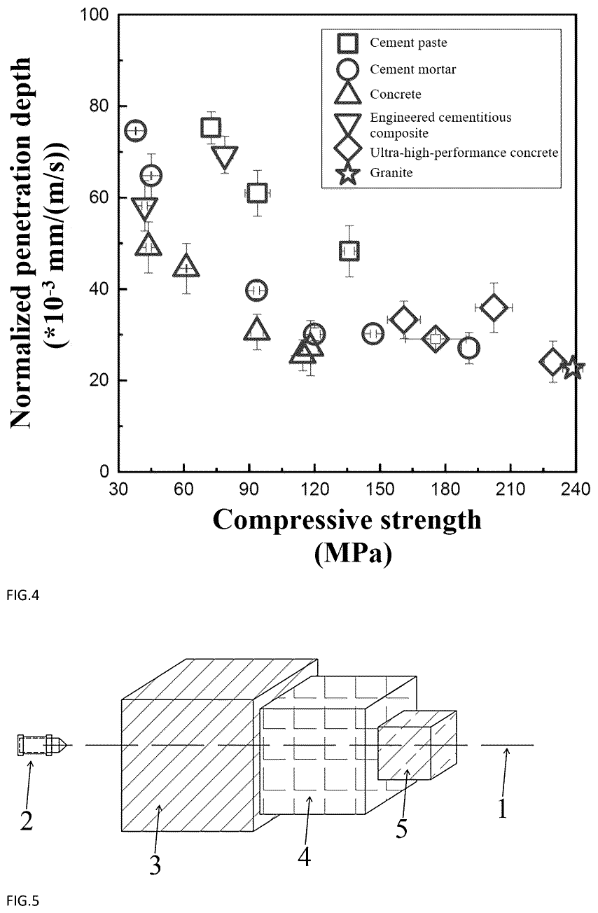

As described in the background, with improvement of the compressive performance, the compressive strength lost a clear association relationship with the penetration resistance, and for example, under the same penetration condition, the UHPC with the compressive strength being 160 MPa to 210 MPa had the similar normalized penetration depth to the concrete with the compressive strength being 90 MPa to 110 MPa, as shown in FIG. 4. Therefore, it urgently needs to provide a method for evaluating the penetration resistance of the cement-based composites capable of comprehensively considering the influence of the material composition.

The method for determining usability of cement-based composites includes:

S1: The cement-based composite with different known components is regarded as a three-phase material consisting of a mortar matrix phase, a coarse aggregate phase, and a fiber phase; the mortar matrix phase includes one or more of cement, silica fume, and fly ash; and the coarse aggregate phase includes a rock aggregate with a particle size of 5 mm to 16 mm, and the fiber phase is one or more of a straight copper-coated steel fiber, a hooked-end copper-coated steel fiber, and an organic fiber.

S2: According to the above method for measuring the Rockwell hardness of the cement-based composite, the Rockwell hardness of the mortar matrix phase is measured; a coarse aggregate raw material is processed into a specimen with the same size as a mortar matrix phase specimen in Example 1, and its Rockwell hardness is measured according to the method in Example 1; and in the method for testing the Rockwell hardness of fiber, by considering the diameter of the fiber is very small (generally 0.2 mm), the Rockwell hardness of all fibers is measured by a fiber base material sample block, and the fiber base material sample block referred to a sample block which had the same material as the fiber phase and might be acquired by cutting a fiber raw material.

S3: The equivalent length of each phase is calculated based on a volume ratio of each phase in the cement-based composite, and this operation includes:

Each constituent phase in the cement-based composite is respectively equivalized into a cube, and the volume of the cube is determined by the volume ratio of each phase; and a cube root is extracted from the volume of the cube to acquire an edge length of the cube to be used as an equivalent length of the phase.

A method for calculating the equivalent length of the fiber phase includes: the volume ratio of the equivalent cube of the fiber phase is multiplied with a correction coefficient k, and then, a cube root is extracted from the volume corrected by the correction coefficient k to obtain the equivalent length of the fiber phase. This is because in the cement-based composites, the differences of the materials, shapes and structures of the fibers might influence the hardness and penetration resistance of the cement-based composites to different degrees, and these influences are difficult to measure by singly considering the volume ratio. Specifically, k is 1.0 in a case that the fiber is a straight copper-coated steel fiber; k is 1.2 in a case that the fiber is a hooked-end copper-coated steel fiber; and k is 0.9 in a case that the fiber is an organic fiber.

S4: Based on the Rockwell hardness of each phase acquired through measurement in S2 and the equivalent length of each phase acquired in S3, weighting is performed to acquire the effective hardness of the cement-based composite; the effective hardness of the cement-based composite is recorded as H_{Effective}, and a weighting calculation method is as follows:

$${H}_{Effective} = \frac{{l}_{Coarse aggregate}}{{l}_{Coarse aggregate} + {l}_{Matrix} + {l}_{Fiber}} {H}_{Coarse aggregate} + \frac{{l}_{Matrix}}{{l}_{Coarse aggregate} + {l}_{Matrix} + {l}_{Fiber}} {H}_{Matrix} + \frac{{l}_{Fiber}}{{l}_{Coarse aggregate} + {l}_{Matrix} + {l}_{Fiber}} {H}_{Fiber} ;$$

wherein,

$${l}_{Coarse aggregate} = \sqrt[3]{{V}_{Coarse aggregate}}$$

is all equivalent length of the coarse aggregate in the cement-based composite;

$${l}_{Matrix} = \sqrt[3]{{V}_{Matrix}}$$

is an equivalent length of the mortar matrix in the cement based composite;

$${l}_{Fiber} = \sqrt[3]{k {V}_{Fiber}}$$

is an equivalent length of the fiber in the cement-based composite, a value of k is selected according to the fiber type, and k is 1.0 in a case that the fiber is a straight copper-coated steel fiber; k is 1.2 in a case that the fiber is a hooked-end copper-coated steel fiber; k is 0.9 in a case that the fiber is an organic fiber;

H_{Coarse aggregate} is a Rockwell hardness value of the coarse aggregate in the cement-based composite;

H_{Matrix} is a Rockwell hardness value of the mortar matrix in the cement-based composite; and

H_{Fiber} is a Rockwell hardness value of the fiber in the cement-based composite.

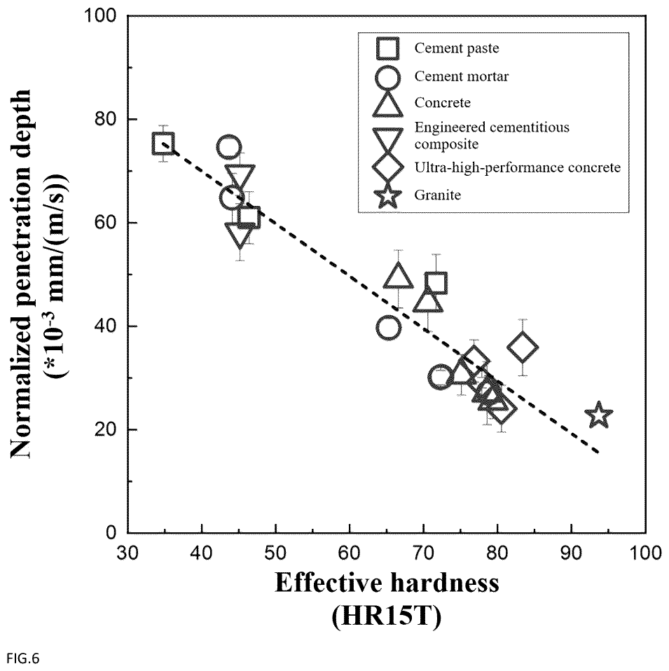

As shown in FIG. 5, according to the method, each dispersed constituent phase of the cement-based composite is respectively concentrated into a cube again, the cubes are sequentially arranged onto a projectile trajectory 1, so when a projectile 2 is in contact with the mortar matrix phase 3 at the most front end, a coarse aggregate phase 4 and a fiber phase 5 arranged at a rear side received force exerted by the projectile 2 at the same time, and multiple constituent phases resisted the impact of the projectile 2 as a whole. For reflection on the calculation of the effective hardness, the effective hardness is acquired by weighting the effective hardness of each constituent phase.

S5: The penetration resistance of the cement-based composite with different components is measured, and an association relationship between the effective hardness and the penetration resistance is established based on the effective hardness corresponding to multiple cement-based composites with different known components acquired in S1 to S4.

Five kinds of cement pastes (serial numbers: CP-1 to CP-5) with different composition ratios are used, values of their effective hardness and penetration resistance are acquired according to the method in Example 1, and their compressive strengths are tested. Since the cement pastes include no coarse aggregate phase and no fiber phase, the effective length of the coarse aggregate phase and the fiber phase is respectively 0.

Seven kinds of cement mortars (serial numbers: CM-1 to CM-7) with different composition ratios are used, values of their effective hardness and penetration resistance are acquired according to the method in Example 1, and their compressive strengths are tested. Since the cement mortars include no coarse aggregate phase and no fiber phase, the effective length of the coarse aggregate phase and the fiber phase is respectively 0.

Seven kinds of concrete materials (serial numbers: CC-1 to CC-7) with different composition ratios are used, values of their effective hardness and penetration resistance are acquired according to the method in Example 1, and their compressive strengths are tested. The components of the concrete include cement mortar and coarse aggregates, but include no fiber, so the effective length of the fiber phase is 0.

Two kinds of high-ductility cement-based composites (serial numbers: ECC-1 to ECC-2) with different composition ratios are used, values of their effective hardness and penetration resistance are acquired according to the method in Example 1, and their compressive strengths are tested. Components of the high-ductility cement-based composites include water, silica fume, fly ash, fiber, a water reducer, and river sand. The river sand is a fine aggregate, and belonged to a composition part of the cement mortar, and a coarse aggregate is not included, so the effective length of the coarse aggregate phase is 0.

Four kinds of ultra-high-performance concretes (serial numbers: UHPC-1 to UHPC-4) with different composition ratios are used, values of their effective hardness and penetration resistance are acquired according to the method in Example 1, and their compressive strengths are tested. Components of the ultra-high-performance concretes include cement mortar matrixes, coarse aggregates and fibers.

Granite used as the coarse aggregate is taken, values of its effective hardness and penetration resistance are acquired according to the method in Example 1, and its compressive strength is tested. The granite includes no cement mortar and no fiber, so the effective length of the matrix phase and the effective length of the fiber phase are 0. A calculation example of the effective hardness is as shown in Table 1.

TABLE 1

| | Rockwell hardness | | | Volume content | | |
|---|---|---|---|---|---|---|
| | Mortar | Coarse | Steel | Mortar | Coarse | Steel |
| | matrix | aggregate | fiber | matrix | aggregate | fiber |
| Concrete C-5 | 69.1 | 93.7 | 80.3 | 64.078% | 35.422% | 0.500% |
| (including coarse | | | | | | |
| aggregate) | | | | | | |

By substituting the data in Table 1 into the weighting calculation method in Example 1, a calculation result of the effective hardness is as follows:

$${H}_{Effective} = \frac{\sqrt[3]{0.35422}}{\sqrt[3]{0.35422} + \sqrt[3]{0.64078} + \sqrt[3]{0.005}} * 93.7 + \frac{\sqrt[3]{0.64078}}{\sqrt[3]{0.35422} + \sqrt[3]{0.64078} + \sqrt[3]{0.005}} * 6 9.1 + \frac{\sqrt[3]{0.005}}{\sqrt[3]{0.35422} + \sqrt[3]{0.64078} + \sqrt[3]{0.005}} = 80.2 .$$

A test method for measuring the penetration resistance of the cement-based composites includes:

S51: A cement-based composite target body is prepared, and the size of the target body is 300×170×150 mm³ (150 mm is the thickness in a direction along the projectile trajectory); and a projectile for the test is prepared, the projectile is a conical projectile with a diameter being 8 mm, and is prepared by ASSAB XW-42 high-strength alloy steel, a yield strength of a projectile material is 2150 MPa to 2200 MPa, a peak strength is 2950 MPa to 3100 MPa, and a Rockwell hardness is 60 HRC to 62 HRC.

S52: A high-speed projectile penetration device is set, projectile trajectory smoothbore cannon is used for launching the projectile, the launching direction is parallel to the thickness direction of the target body along the projectile trajectory, and a penetration crater is in the center position of the target body plane. In the process, the speed of the projectile is measured before and after the launching using a laser speed measuring system, and in addition, whether the direction of the projectile trajectory is perpendicular to the target body or not is monitored using a high-speed camera so as to eliminate an atypical test result caused by launching angle deviation.

S53: An anti-penetration experiment is performed, the damage degree of the target body of the cement-based composite under the action of the high-speed projectile at the set projectile speed is measured through the penetration depth (a penetration distance of the projectile through the target body to the deepest point) and the crater diameter (the diameter of an equivalent circle with the same area with a projectile crater).

The normalized penetration depth Y_{Penetration depth} is calculated through the penetration depth. A calculation method is as follows: the normalized penetration depth=penetration depth/target impact velocity. The target impact velocity is acquired by measuring the projectile speed after launching using the laser speed measuring system.

Through the practical test, the compressive strength, the normalized penetration depth and the crater diameter of each material are acquired, and are summarized with the effective hardness, as shown in Table 2.

TABLE 2

| | Compressive | Effective | Normalized | Crater |
|---|---|---|---|---|
| Serial | strength | hardness | penetration depth | diameter |
| number | (MPa) | (HR15T) | (*10⁻³ mm/(m/s)) | (mm) |
| CP-1 | 70.9 | 34.8 | 75.3 | 75.9 |
| CP-2 | 90.4 | 46.4 | 61.0 | 72.9 |
| CP-3 | 100.3 | 52.1 | 48.3 | 66.2 |
| CM-1 | 34.2 | 43.7 | 74.6 | 66.2 |
| CM-2 | 40.3 | 44.1 | 64.8 | 60.7 |
| CM-3 | 88.0 | 65.3 | 39.7 | 59.3 |
| CM-4 | 120.1 | 72.3 | 30.1 | 53.9 |
| CM-5 | 142.8 | 72.4 | 30.2 | 55.8 |
| CM-6 | 189.7 | 78.8 | 27.1 | 51.8 |
| CC-1 | 40.9 | 66.6 | 49.1 | 60.6 |
| CC-2 | 49.2 | 69.4 | 37.3 | 58.5 |
| CC-3 | 57.4 | 70.6 | 44.5 | 56.8 |
| CC-4 | 87.7 | 75.1 | 30.6 | 53.3 |
| CC-5 | 113.1 | 79.4 | 25.5 | 53.0 |
| CC-6 | 122.0 | 78.6 | 27.1 | 50.4 |
| CC-7 | 158.2 | 83.4 | 24.3 | 52.1 |
| CC-8 | 197.2 | 84.7 | 20.2 | 49.2 |
| ECC-1 | 37.9 | 45.2 | 58.2 | 43.9 |
| ECC-2 | 76.6 | 45.2 | 69.4 | 46.3 |
| UHPC-1 | 156.3 | 76.4 | 33.3 | 50.2 |
| UHPC-2 | 168.1 | 77.8 | 29.1 | 46.7 |
| UHPC-3 | 202.3 | 83.4 | 35.5 | 54.9 |
| UHPC-4 | 220.2 | 79.2 | 24.1 | 49.1 |
| Granite | 238.5 | 93.7 | 22.7 | 37.4 |

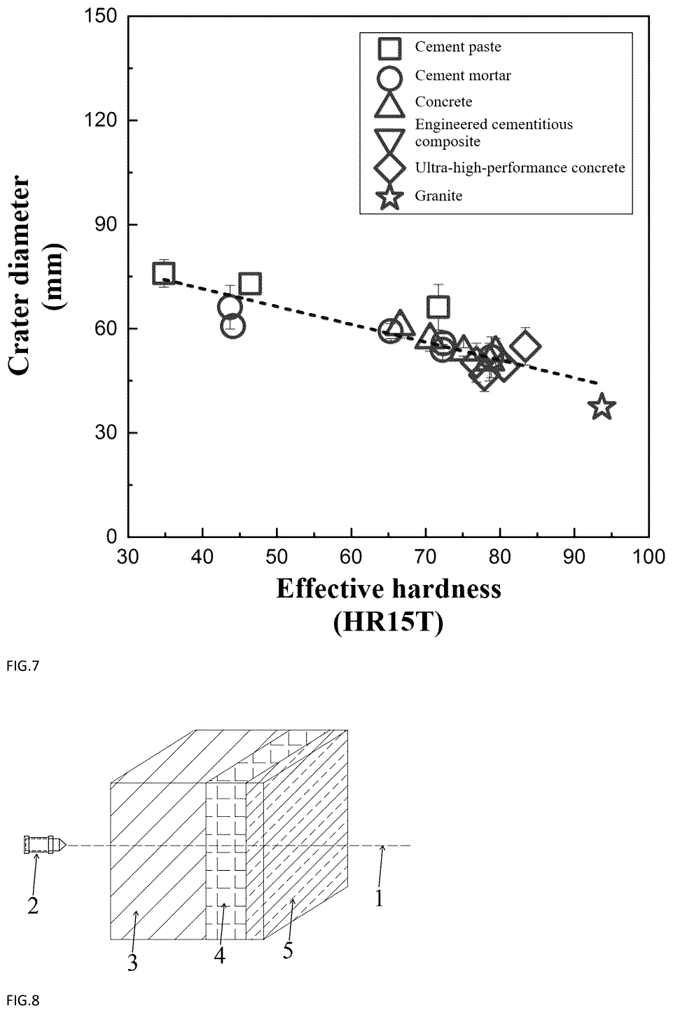

The data statistical processing is performed based on data in Table 2 to acquire a diagram of a relationship between the effective hardnesses and the normalized penetration depths as shown in FIG. 6 and a diagram of a relationship between the effective hardnesses and the crater diameters as shown in FIG. 7. It could be seen that there is a clear linear relationship between the effective hardness and the penetration resistance, and an evaluation value could be acquired based on the effective hardness.

Through sorting, the linear relationship between the effective hardness and the normalized penetration depth is as shown by a dotted line in FIG. 6, and a calculation method of the evaluation value “Y_{Evaluation-Penetration depth}” of the normalized penetration depth is as follows:

$${Y}_{Evaluation - Penetration depth} = - 0.411 {H}_{Effective} + 45.004 ; {R}^{2} = 0 \text{.91} ;$$

the linear relationship between the effective hardness and the crater diameter is as shown by a dotted line in FIG. 7, and a calculation method of the evaluation value “Y_{Evaluation-Crater diameter}” of the crater diameter is as follows:

$${Y}_{Evaluation - Crater diameter} = - 0.513 {H}_{Effective} + 92.016 ; {R}^{2} = 0.80 .$$

According to Table 2, it could be known that the compressive strength of the cement participating in fitting and the material is 30 MPa to 240 MPa.

Values of the effective hardnesses of cement-based composites with the unknown penetration resistance are acquired according to the method of the present example. The effective hardness of each material is acquired, and is substituted into the method for calculating the penetration depth and the crater diameter in the present example to solve the penetration depth Y_{Evaluation-Penetration depth} acquired through evaluation and the crater diameter Y_{Evaluation-Crater diameter} acquired through evaluation.

S6: The cement-based composite with unknown penetration resistance is regarded as a multiphase material, the effective hardness of the cement-based composite with unknown penetration resistance is calculated according to S1 to S4, and calculation is performed using the association relationship acquired in S5 to acquire an evaluation value of the normalized penetration depth and an evaluation value of the crater diameter of the cement-based composite with unknown penetration resistance.

S7: Corresponding threshold ranges of the acquired evaluation value are judged, and the cement-based composite is applied to intended use thereof.

According to different evaluation results, each material could be applied to its intended building with different anti-penetration requirements. In a case that the evaluation value of the normalized penetration depth is below 10.069 mm, and the evaluation value of the crater diameter is below 48.411 mm, the material could be used for a missile launching silo. In a case that the evaluation value of the normalized penetration depth is 10.069 mm to 22.399 mm, and the evaluation value of the crater diameter is 48.411 mm to 63.801 mm, the material could be used for an airport runway. In a case that the evaluation value of the normalized penetration depth is above 22.399 mm, and the evaluation value of the crater diameter is above 63.801 mm, the material could be used for an ordinary shelter.

### Comparative Example

The present comparative example provided another method for calculating the effective hardness. It differed from Example 1 in that: a method for acquiring the effective length includes:

As shown in FIG. 8, each constituent phase in the cement-based composite is respectively equivalized into a cuboid with the same cross section and different thicknesses, the volumes of the cuboids are determined based on the volume ratio of each phase, the phases include a mortar matrix phase 3, a coarse aggregate phase 4 and a fiber phase 5 in sequential arrangement, each cuboid is sequentially arranged on a projectile trajectory 1, and multiple constituent phases resisted the impact of a projectile 2 as a whole. The thickness acquired by dividing the volume of each cuboid by the same cross section is used as the equivalent length of the phase, i.e., the equivalent length is in a directly proportional relationship with the volume of each phase rather than a square root relationship, and the acquired effective hardness is recorded as H_{Comparative}.

Through data processing, it could be known that there is no clear linear relationship between value relationship of H_{Comparative} and the penetration resistance, so the penetration resistance of the cement-based composite could not be evaluated using the method for acquiring the effective length in the Comparative example.

The penetration resistance of the sample in the present example practically measured according to the method in Example 1, and the evaluation value

“Y_{Evaluation-Penetration depth}” of the normalized penetration depth and the evaluation value “Y_{Evaluation-Crater diameter}” of the crater diameter acquired through calculation had good conformance condition with the practically measured data. It showed that the method for evaluating the penetration resistance acquired by the present example could be applied to the fiber-reinforced cement-based composite with the unknown performance.

It showed that the present invention used the effective hardness as a new index for measuring the penetration resistance of the cement-based composite. The present example showed that with the improvement of the effective hardness, the penetration resistance of the material is correspondingly enhanced. It showed that the method for evaluating the penetration resistance of the cement-based composite provided by the present invention could provide important theoretical support and practice guidance for further study and development of the cement-based composite.

The foregoing descriptions are merely exemplary examples of the present invention, but are not intended to limit the present invention. A person skilled in the art may make various alterations and variations to the present invention. Any modification, equivalent replacement, improvement, or the like made within the spirit and principle of the present invention shall fall within the protection scope of the present invention.

1. A method for determining usability of cement-based composites, comprising:
S1: regarding a cement-based composite with different known components as a multiphase material, and dividing each component of the cement-based composite into a plurality of constituent phases;
S2: acquiring samples of all constituent phases, and respectively measuring a Rockwell hardness of each phase;
S3: calculating an equivalent length of each phase based on a volume ratio of each phase in the cement-based composite;
S4: acquiring an effective hardness of the cement-based composite through weighted calculation based on the Rockwell hardness of each phase acquired through measurement in S2 and the equivalent length of each phase acquired in S3;
S5: measuring penetration resistance of the cement-based composite with different components, and establishing an association relationship between the effective hardness and the penetration resistance based on the effective hardness corresponding to a plurality of cement-based composites with different known components acquired in S1 to S4;
S6: regarding a cement-based composite with unknown penetration resistance as a multiphase material, calculating an effective hardness of the cement-based composite with unknown penetration resistance according to S1 to S4, and performing calculation using the association relationship acquired in S5 to acquire an evaluation value of the penetration resistance of the cement-based composite with unknown penetration resistance; and
S7: judging a corresponding threshold range of the acquired evaluation value, and applying the cement-based composite to intended use thereof.

2. The method for determining usability of cement-based composites according to claim 1, wherein in S1, the cement-based composite is used as a three-phase material consisting of a mortar matrix phase, a coarse aggregate phase, and a fiber phase; the mortar matrix phase comprises one or more of cement, silica fume, and fly ash; and the coarse aggregate phase comprises a rock aggregate with a particle size of 5 mm to 16 mm, and the fiber phase is one or more of a straight copper-coated steel fiber, a hooked-end copper-coated steel fiber, and an organic fiber.

3. The method for determining usability of cement-based composites according to claim 1, wherein in S2, a sample of the cement-based composite is acquired, and a Rockwell hardness on a surface of the sample is measured to be used as a Rockwell hardness of the mortar matrix phase; a coarse aggregate sample in the cement-based composite is acquired, and a Rockwell hardness on a surface of the coarse aggregate sample is measured; and a fiber sample in the cement-based composite is acquired, and a Rockwell hardness of the fiber sample is measured.

4. The method for determining usability of cement-based composites according to claim 3, wherein in S2, a method for measuring the Rockwell hardness of the mortar matrix phase comprises:
S21: casting the cement-based composite to acquire a sample block, the sample block having a plurality of side surfaces adjacent to a cast surface;
S22: grinding the plurality of side surfaces to a smooth state, and then, selecting square measuring regions far away from edges in the side surfaces;
S23: uniformly taking points in the measuring regions, testing the Rockwell hardness, and reading indentation data of a plurality of effective points; and
S24: taking an average value of the indentation data of the plurality of effective points as the Rockwell hardness of the cement-based composite, wherein:
a method for judging effective points comprises: enabling a distance between indentation centers of adjacent effective points not to be smaller than 4 times of a diameter of a spherical indenter used in a Rockwell hardness measuring process, and enabling a distance between the indentation center and the edge of the sample not to be smaller than 2.5 times of the diameter of the used spherical indenter.

5. The method for determining usability of cement-based composites according to claim 4, wherein in S21, the sample block is a cube, the cast surface is any one surface of the cube, and the sample block has four side surfaces adjacent to the cast surface;
or, in S21, the sample block is subjected to demoulding, grinding, and cleaning to acquire a sample to be tested, and the sample to be tested is a cube block being 100×100×100 mm³;
or, in S22, a plurality of edge perpendicular lines are drawn on the side surfaces to uniformly divide each side surface into a plurality of small square regions, and selecting the square regions in center positions as the measuring regions;
or, in S23, the points are taken in the center positions of the small square regions;
or, in S24, at least 100 effective points are selected for reading from the three cube sample blocks each with the size of 100×100×100 mm³.

6. The method for determining usability of cement-based composites according to claim 3, wherein in S2, a method for measuring the Rockwell hardness of the coarse aggregate comprises: processing a coarse aggregate raw material to present the same shape as the sample of the cement-based composite, and then, performing measurement according to the method for measuring the Rockwell hardness of the mortar matrix phase.

7. The method for determining usability of cement-based composites according to claim 1, wherein in S3, an equivalent length of a mortar matrix is acquired based on a volume ratio of the mortar matrix phase, the coarse aggregate phase, and the fiber phase; an equivalent length of a coarse aggregate is acquired; and an equivalent length of a fiber is acquired.

8. The method for determining usability of cement-based composites according to claim 1, wherein in S3, the equivalent length of each phase is solved by a cube root of a volume fraction of each phase, and the equivalent length of the fiber phase is corrected according to a fiber type;
or, in S3, a method for calculating the equivalent lengths of the coarse aggregate phase, the mortar matrix phase, and the fiber phase comprises:
$${l}_{Coarse aggregate} = \sqrt[3]{{V}_{Coarse aggregate}} ; \\ {l}_{Matrix} = \sqrt[3]{{V}_{Matrix}} ; \\ {l}_{Fiber} = \sqrt[3]{k {V}_{Fiber}} ; \\ {V}_{Coarse aggregate} + {V}_{Matrix} + {V}_{Fiber} = 100 \% ,$$
wherein:
V_{Coarse aggregate} is a volume ratio of the coarse aggregate phase in the cement-based composite;
V_{Matrix} is a volume ratio of the mortar matrix phase in the cement-based composite; and
V_{Fiber} is a volume ratio of the fiber phase in the cement-based composite;
wherein, a value of k is selected according to the fiber type, and k is 1.0 in a case that the fiber is a straight copper-coated steel fiber; k is 1.2 in a case that the fiber is a hooked-end copper-coated steel fiber; and k is 0.9 in a case that the fiber is an organic fiber.

9. The method for determining usability of cement-based composites according to claim 8, wherein in S4, in a weighting calculation method, a weight of the Rockwell hardness of each phase equals to a ratio of the equivalent length of the phase to a sum of the equivalent lengths of all phases; and
by recording the effective hardness of the cement-based composite as H_{Effective}, the weighting calculation method is as follows:
$${H}_{Effective} = \frac{{l}_{Coarse aggregate}}{{l}_{Coarse aggregate} + {l}_{Matrix} + {l}_{Fiber}} {H}_{Coarse aggregate} + \frac{{l}_{Matrix}}{{l}_{Coarse aggregate} + {l}_{Matrix} + {l}_{Fiber}} {H}_{Matrix} + \frac{{l}_{Fiber}}{{l}_{Coarse aggregate} + {l}_{Matrix} + {l}_{Fiber}} {H}_{Fiber} ;$$
wherein:
H_{Coarse aggregate} is a Rockwell hardness value of the coarse aggregate in the cement-based composite;
H_{Matrix} is a Rockwell hardness value of the mortar matrix in the cement-based composite;
and H_{Fiber} is a Rockwell hardness value of the fiber in the cement-based composite.

10. The method for determining usability of cement-based composites according to claim 1, wherein in S5, the association relationship comprises: a method for calculating an evaluation value of a normalized penetration depth is as follows:
$${Y}_{Evaluation - Penetration depth} = - 0.411 {H}_{Effective} + 45.004 ; {R}^{2} = 0 \text{.91} ;$$
and, a method for calculating an evaluation value of a crater diameter is as follows:
$${Y}_{Evaluation - Crater diameter} = - 0.513 {H}_{Effective} + 92.016 ; {R}^{2} = 0.80 .$$
